(19) United States
(12) Patent Application Publication
(10) Pub. No.: US 2010/0204507 A1
(43) Pub. Date: Aug. 12, 2010

(54) ACYLOXY COMPOUNDS OF ELEMENTS OF THE BORON GROUP

(76) Inventors: Jens Röder, Goslar (DE); Dirk Dawidowski, Friedberg (DE)

Correspondence Address: FULBRIGHT & JAWORSKI, LLP, 666 FIFTH AVE, NEW YORK, NY, 10103-3198, US

(21) Appl. No.: 12/674,779

(22) PCT Filed: Sep. 1, 2008
(86) PCT No.: PCT/EP08/07126

§ 371 Date: Apr. 23, 2010

(30) Foreign Application Priority Data
Aug. 31, 2007 (DE) 10 2007 041 443.0

(51) Int. Cl. C07C 69/604

(52) U.S. Cl. 560/190

(57) ABSTRACT

The present invention relates to acyloxy compounds of elements of the boron group, the production of said compounds and the use thereof.

The present invention provides acyloxy compounds of elements of the boron group, the preparation of these compounds, and the use thereof in organic synthesis.

In particular, the invention provides acyloxy compounds of boron or of aluminium.

The use of sodium cyanoborohydride Na[BH₃CN] as reagent for reductive amination is sufficiently well-known. By reason of the toxicity of this compound, substitutes have been sought. It is known that sodium triacetoxyborohydride Na[(CH₃COO)₃BH], just like Na[BH₃CN], is a selective hydrogenation reagent for ketones, aldehydes, halides for diastereoselective reduction and for reductive amination. A disadvantageous aspect of the use of Na[(CH₃COO)₃BH] is, above all, its state of aggregation. The fine dust is difficult to handle. Production technicians have to protect themselves against the fine dust by means of special personal protective equipment, or have to handle the substance totally within glove boxes. But, above all, Na[(CH₃COO)₃BH] is sparingly soluble or not soluble at all in most standard solvents that are suitable for synthesis. Na[(CH₃COO)₃BH] is virtually insoluble in hydrocarbons and in diethyl ether. In tetrahydrofuran the solubility amounts to merely 2%; in dimethoxyethane it amounts to less than 5%. The synthesis of Na[(CH₃COO)₃BH] is comparatively elaborate. In order to cause three H atoms of the initial substance NaBH₄ to react, long reaction-times and a great excess of acetic acid are necessary. This excess must, in turn, be washed out of the solid in an elaborate manner. An overview of the state of the art is given by: A. F. Abdel-Magid, C. A. Maryanoff “Reductions in Organic Synthesis: Recent Advances and Practical Applications”, A. F. Abdel-Magid, ed. American Chemical Society, Washington, D.C., 1996, pp. 201-216; J. Seyden-Penne “Reductions by the Alumino- and Borohydrides in Organic Synthesis”, VCH Publishers, INC./Lavoisier—Tec & Doc: New York, N.Y., 1991, pp. 44, 66.

As an alternative to Na[(CH₃COO)₃BH], other triacyloxyborohydrides have already been developed. Known are the compounds of trifluoroacetic acid, propionic acid, isobutyric acid, pivalic acid, butyric acid, valeric acid, caproic acid, cyclohexanoic acid, 2-ethylbutyric acid, 2-ethylhexanoic acid and benzoic acid. These triacyloxyborohydrides with longer acyloxy substituents are distinguished by a better solubility in organic solvents and, for the most part, a higher selectivity in the course of stereoselective syntheses. However, the rate of reaction is, for the most part, slower compared with Na[BH₃CN] or Na[(CH₃COO)₃BH] (E. R. Burkhardt, K. Matos, Chem. Rev. 2006, 106, 2617-2650; J. M. McGill, E. S. LaBell, M. Williams, Tetrahedron Lett. 1996, 3977-3980). The acid residues that are used also have considerable disadvantages: trifluoroacetic acid is highly toxic; as is generally known, the low-molecular acids smell disgusting, impeding especially use of butyric acid and of its singly substituted derivatives on a technical scale. Likewise it is known that 2-ethylhexanoic acid is suspected of possessing teratogenic properties and of harming the unborn baby. The benzoic acid derivative additionally has the undesirable property of reducing itself to alcohol (G. W. Gribble, C. F. Nutaitis, Organic Preparations and Procedures Int., 1985, 17, 317-384).

Ane object of the present invention is to overcome the disadvantages of the prior art.

In particular, the object of the present invention is to make available compounds that are selective hydrogenation reagents for ketones, aldehydes and/or halides and/or that can be employed for diastereoselective reduction and/or reductive amination. These compounds are to be readily soluble in organic solvents, are to exhibit no toxicity or only slight toxicity, are to contain no fluorine substituents, are to be easy to synthesise and/or are to exhibit only a slight inherent odour.

In accordance with the invention the object is achieved by the features of the main claim. Preferred configurations are to be found in the dependent claims.

In accordance with the invention the object is surprisingly achieved by acyloxy compounds of elements of the boron group, the acyloxy group possessing three further substituents on the α carbon atom that are different from H. The object is preferentially achieved, in accordance with the invention, by acyloxy compounds of the boron group of the general formula 1 represented below:

M⁺[(R¹R²R³CCOO)₃XH]⁻ (Formula 1),

where:

X is a trivalent element of the boron group, in accordance with the invention preferably boron and aluminium, in accordance with the invention particularly preferably boron;

R¹, R², R³ are selected, independently of one another, from functionalised and/or unfunctionalised branched and/or unbranched alkyl, alkenyl, alkinyl, cycloalkyl groups with 1 to 20 C atoms and/or aryl groups with 1 to 12 C atoms;

M⁺ is an alkali metal, Li, Na, K, Rb, Cs or [(R⁴R⁵R⁶R⁷)N]⁺ or H⁺ or [(C₆H₅)₃C]⁺ or mixtures thereof;

R⁴, R⁵, R⁷ are selected, independently of one another, from H, functionalised and/or unfunctionalised branched and/or unbranched alkyl, alkenyl, alkinyl, cycloalkyl groups with 1 to 20 C atoms and/or aryl groups with 1 to 12 C atoms.

In accordance with the invention the sodium cation is preferred by way of M⁺.

Examples of R¹, R² and R³ are: methyl, ethyl, ethenyl, ethinyl, n-propyl, isopropyl, cyclopropyl, propen-3-yl, propin-3-yl, n-butyl, cyclobutyl, 1-buten-4-yl, 1-butin-4-yl, 2-buten-4-yl, crotyl, 2-butin-4-yl, 2-butyl, isobutyl, tert-butyl, n-pentyl, cyclopentyl, cyclopentadienyl, isopentyl, neopentyl, tert-pentyl, cyclohexyl, hexyl, n-heptyl, isoheptyl, n-octyl, iso-octyl, thexyl, 2-ethyl-1-hexyl, 2,2,4-trimethylpentyl, nonyl, decyl, dodecyl, n-dodecyl, cyclopentyl, cyclopentenyl, cyclohexyl, cyclohexenyl, cycloheptyl, methylcyclohexyl, naphthyl, anthranyl, phenanthryl, o-tolyl, p-tolyl, m-tolyl, xylyl, ethylphenyl, mesityl, phenyl, benzyl, trimethylsilyl, triisopropylsilyl, tri-tert-butylsilyl, dimethylthexylsilyl, 1,1,1,3,3,3-heptamethyl-2-(trimethylsilyl)trisilan-2-yl, 1,1,1-tris(trimethylsilyl)methyl, trimethylsilylethinyl, triisopropylsilylethinyl, tri-tert-butylsilylethinyl, dimethylthexylsilylethinyl.

Preferred in accordance with the invention are carboxylic acid anions in which the total number of carbon atoms of the residues R¹, R² and R³ is equal to 4. Particularly preferably, R¹═R²=methyl and R³=ethyl. An appropriate carboxylic acid for this, which is substituted in such a manner on the α carbon atom, is available from Brenntag under the name Versatic® 6. Further preferred in accordance with the invention are compounds in which the total number of carbon atoms of the residues R¹, R² and R³ is equal to 6. Particularly preferred in accordance with the invention are compounds in which the total number of carbon atoms of the residues R¹, R² and R³ is equal to 8. An appropriate carboxylic acid for this, which is substituted in such a manner on the a carbon atom, is available from Brenntag under the name neodecanoic acid or Versatic® 10.

Examples of R⁴, R⁵, R⁶ and R⁷ are: H, methyl, ethyl, ethenyl, ethinyl, n-propyl, isopropyl, cyclopropyl, propen-3-yl, propin-3-yl, n-butyl, cyclobutyl, 1buten-4-yl, 1-butin-4-yl, 2-buten-4-yl, crotyl, 2-butin-4-yl, 2-butyl, isobutyl, tert-butyl, n-pentyl, cyclopentyl, cyclopentadienyl, isopentyl, neopentyl, tert-pentyl, cyclohexyl, hexyl, n-heptyl, isoheptyl, n-octyl, iso-octyl, thexyl, 2-ethyl-1-hexyl, 2,2,4-trimethylpentyl, nonyl, decyl, dodecyl, n-dodecyl, cyclopentyl, cyclopentenyl, cyclohexyl, cyclohexenyl, cycloheptyl, methylcyclohexyl, naphthyl, anthranyl, phenanthryl, o-tolyl, p-tolyl, m-tolyl, xylyl, ethylphenyl, mesityl, phenyl, benzyl.

Preferred in accordance with the invention are compounds in which Formula 1 stands for Na[(neodecanoate)₃BH], Na[(CH₃CH₂(CH₃)₂COO)₃BH] or (Na[(Versatate-6)₃BH]).

The acyloxy compounds of elements of the boron group according to the invention are surprisingly distinguished by a good solubility in organic solvents and by an unobtrusive odour; they contain no fluorine substituents, and no great excess of the carboxylic acid is necessary for their synthesis.

The invention further provides:
the application of the acyloxy compounds of elements of the boron group according to the invention in organic synthesis;
the application of the acyloxy compounds of elements of the boron group according to the invention as reducing agents;
the application of the acyloxy compounds of elements of the boron group according to the invention as reducing agents in the presence of a carboxylic acid. The ratio of the carboxylic acid in these applications to the acyloxy compounds of the boron group according to the invention may amount to from 0:1 to 10:1, particularly preferably from 1:1 to 2:1. A carboxylic acid that is particularly preferred in accordance with the invention is acetic acid. Water may be added to the reduction mixture.
the application of the acyloxy compounds of elements of the boron group according to the invention as reducing agents for oxo compounds such as ketones and/or aldehydes and/or esters and the nitrogen analogues thereof, the imines;
the application of the acyloxy compounds of elements of the boron group according to the invention for reductive amination of oxo compounds such as ketones and/or aldehydes;
the application of the acyloxy compounds of elements of the boron group according to the invention for the reduction of halides;
the application of the acyloxy compounds of elements of the boron group according to the invention as stereoselective reducing agents.

A solvent may be added to the acyloxy compounds of elements of the boron group according to the invention.

Surprisingly, the acyloxy compounds according to the invention are superior to the known acyloxy compounds in their designated application.

The synthesis of the acyloxy compounds of elements of the boron group according to the invention may be undertaken in a solvent. The proportion of the solvent in the course of the synthesis preferably amounts to 0% to 70%, preferably 20% to 55%, particularly preferably 30% to 45%.

Preferred solvents are alicyclic ethers, aliphatic and aliphatic difunctional ethers, esters, ketones, carbonates, nitriles, amines, acid amides, ionic liquids, water, alcohols, hydrocarbons, halogenated hydrocarbons, heterocyclic compounds and heteroaromatic compounds or mixtures of at least two such solvents. Particularly preferred are diethyl ether, tetrahydrofuran, tetrahydro-2-methylfuran, tetrahydro-3-methylfuran, tetrahydro-2,5-dimethylfuran, tetrahydro-3,4-dimethylfuran, tetrahydropyran, cyclopentyl methyl ether, dimethoxyethane, diethoxymethane, diethoxyethane, polyethylene glycols, methanol, ethanol, propanol, isopropanol, butanol, isobutanol, tert-butanol, sec-butanol, 2-ethylhexanol, methyl formate, ethyl formate, propyl formate, methyl isobutyl ketone, methyl acetate, ethyl acetate, butyl acetate, methoxyethyl acetate, ethoxyethyl acetate, 2-(2-ethoxyethoxy)ethyl acetate, (2-methoxyethyl)methyl carbonate, ethylene carbonate, propylene carbonate, butylene carbonate, vinylene carbonate, dimethyl carbonate, diethyl carbonate, dipropyl carbonate, ethylmethyl carbonate, methyl propyl carbonate, butylmethyl carbonate, ethylpropyl carbonate, butylethyl carbonate, γ-butyrolactone, γ-valerolactone, dimethylformamide, dimethylacetamide, dimethyl sulfoxide, triethylamine, piperidine, pyridine, acetonitrile, propionitrile, glutarodinitrile, adiponitrile, methoxypropionitrile, pentane, hexane, cyclohexane, heptane, methylcyclohexane, octane, benzene, toluene, xylenes (all isomers), ethylbenzene, cumene, dichloromethane, chloroform, 1,2-dichloroethane or mixtures of at least two such solvents.

The acyloxy compounds of elements of the boron group according to the invention are generally prepared in accordance with the following synthesis specification:

One equivalent of a suitable compound of an element of the boron group is converted in a suitable solvent with 1 to 6, preferably with 2 to 4, particularly preferably with 2.7 to 3.3, equivalents of a carboxylic acid corresponding to the carboxylic acid anion R¹R²R³CCOO″ according to the invention. The suitable compound of an element of the boron group may, for example, have been selected from Li[BH₄], Li[AlH₄], Na[BH₄] or Na[AlH₄]. Preferred in accordance with the invention are Na[BH₄] and Li[AlH₄]; particularly preferred in accordance with the invention is Na[BH₄]. Suitable solvents are alicyclic ethers, aliphatic and aliphatic difunctional ethers, esters, ketones, carbonates, nitriles, amines, acid amides, ionic liquids, water, alcohols, hydrocarbons, halogenated hydrocarbons, heterocyclic compounds and heteroaromatic compounds.

A monitoring of the reaction may be undertaken by means of IR spectroscopic tracking or turbidity measurement or particle-size measurement or conductivity measurement directly in the reaction vessel.

The following Examples are intended to elucidate the invention in more detail without thereby restricting it:

### EXAMPLE 1

### Synthesis of Na[(CH₃CH₂(CH₃)₂COO)₃BH]

One equivalent of NaBH₄ is suspended in THF. At 20° C. three equivalents of CH₃CH₂(CH₃)₂COOH (Versatic® 6) are added. Stirring is effected at 20° C. until the evolution of hydrogen is concluded.

### EXAMPLE 2

### Synthesis of Na[(neodecanoate)₃BH]

One equivalent of NaBH₄ is suspended in THF. At 20° C. three equivalents of neodecanoic acid (Versatic®10) are added. Stirring is effected at 20° C. until the evolution of hydrogen is concluded.

### EXAMPLE 3

### Reductive Amination of Cyclohexanone and Benzylamine with Na[(CH₃CH₂(CH₃)₂COO)₃BH]

One equivalent each of cyclohexanone and benzylamine are stirred into THF at 20° C. After the addition of one equivalent of acetic acid, two equivalents of Na[(CH₃CH₂(CH₃)₂COO)₃BH] dissolved in THF are added. After two minutes the reduction is concluded (GC).

### EXAMPLE 4

### Reductive Amination of Cyclohexanone and Aniline with Na[(CH₃CH₂(CH₃)₂COO)₃BH]

One equivalent each of cyclohexanone and aniline are stirred into THF at 20° C. After the addition of one equivalent of acetic acid, two equivalents of Na[(CH₃CH₂(CH₃)₂COO)₃BH] dissolved in THF are added. After 180 minutes a 65% conversion has been attained (GC).

### EXAMPLE 5

### (Comparative Example) Synthesis of Na[(2-ethylhexanoate)₃BH]

One equivalent of NaBH₄ is suspended in THF. At 20° C. three equivalents of 2-ethylhexanoic acid are added. Stirring is effected at 20° C. until the evolution of hydrogen is concluded.

### EXAMPLE 6

### (Comparative Example) Reductive Amination of Cyclohexanone and Aniline with Na[(2-ethylhexanoate)₃BH]

One equivalent each of cyclohexanone and aniline are stirred into THF at 20° C. After the addition of one equivalent of acetic acid, two equivalents of Na[(2-ethylhexanoate)₃BH] dissolved in THF are added. After 180 min a 55% conversion has been attained (GC).

1-19. (canceled)

20. An acyloxy compound of formula
M⁺[(R¹R²R³CCOO)₃XH (Formula 1)
wherein M
⁺ is an alkali metal, Li, Na, K, Rb, Cs or [(R⁴R⁵R⁶R⁷)N]⁺ or H⁺ or [(C₆H₅)₃C]⁺ or mixtures thereof;
X is a trivalent element of the boron group;
the three substituents R¹R²R³ on an α carbon atom of the acyloxy group are different from H; and
R⁴, R⁵, R⁶, R⁷ are independently selected from H, functionalized, unfunctionalized branched, or unbranched alkyl, alkenyl, alkinyl, cycloalkyl groups with 1 to 20 C atoms or aryl groups with 1 to 12 C atoms.

21. An acyloxy compound according to claim 20, wherein R¹, R², R³ are independently selected from functionalized, unfunctionalized, branched or unbranched alkyl, alkenyl, alkinyl, cycloalkyl groups with 1 to 20 C atoms or aryl groups with 1 to 12 C atoms.

22. An acyloxy compound according to claim 20, wherein R¹, R² and R³ are independently selected from the group consisting of methyl, ethyl, ethenyl, ethinyl, n-propyl, isopropyl, cyclopropyl, propen-3-yl, propin-3-yl, n-butyl, cyclobutyl, 1-buten-4-yl, 1-butin-4-yl, 2-buten-4-yl, crotyl, 2-butin-4-yl, 2-butyl, isobutyl, tert-butyl, n-pentyl, cyclopentyl, cyclopentadienyl, isopentyl, neopentyl, tert-pentyl, cyclohexyl, hexyl, n-heptyl, isoheptyl, n-octyl, iso-octyl, thexyl, 2-ethyl-1-hexyl, 2,2,4-trimethylpentyl, nonyl, decyl, dodecyl, n-dodecyl, cyclopentyl, cyclopentenyl, cyclohexyl, cyclohexenyl, cycloheptyl, methylcyclohexyl, naphthyl, anthranyl, phenanthryl, o-tolyl, p-tolyl, m-tolyl, xylyl, ethylphenyl, mesityl, phenyl, benzyl, trimethylsilyl, triisopropylsilyl, tri-tert-butylsilyl, dimethylthexylsilyl, 1,1,1,3,3,3-heptamethyl-2-(trimethylsily)trisilan-2-yl, 1,1,1-tris(trimethylsilyl)methyl, trimethylsilylethinyl, triisopropylsilylethinyl, tri-tert-butylsilylethinyl and dimethylthexylsilylethinyl.

23. An acyloxy compound according to claim 20, wherein R⁴, R⁵, R⁶ and R⁷ independently selected from the group consisting of H, methyl, ethyl, ethenyl, ethinyl, n-propyl, isopropyl, cyclopropyl, propen-3-yl, propin-3-yl, n-butyl, cyclobutyl, 1-buten-4-yl, 1-butin-4-yl, 2-buten-4-yl, crotyl, 2-butin-4-yl, 2-butyl, isobutyl, tert-butyl, n-pentyl, cyclopentyl, cyclopentadienyl, isopentyl, neopentyl, tert-pentyl, cyclohexyl, hexyl, n-heptyl, isoheptyl, n-octyl, iso-octyl, thexyl, 2-ethyl-1-hexyl, 2,2,4-trimethylpentyl, nonyl, decyl, dodecyl, n-dodecyl, cyclopentyl, cyclopentenyl, cyclohexyl, cyclohexenyl, cycloheptyl, methylcyclohexyl, naphthyl, anthranyl, phenanthryl, o-tolyl, p-tolyl, m-tolyl, xylyl, ethylphenyl, mesityl, phenyl and benzyl.

24. An acyloxy compound according to claim 20, wherein R¹, R² and R³ are selected from carboxylic acid anions in which the total number of carbon atoms of the residues R¹, R² and R³ is 4, 6 or 8.

25. An acyloxy compound according to claim 20, wherein R¹ and R² are methyl and R³ is ethyl.

26. An acyloxy compound according to claim 20, wherein M is sodium.

27. An acyloxy compound according to claim 20 that is Na[(neodecanoate)₃BH], Na[(CH₃CH₂(CH₃)₂COO)₃BH] or (Na[(Versatate-6)₃BH]).

28. A process for preparing an acyloxy compound according to claim 20 comprising converting 1 equivalent of a suitable compound of an element of the boron group, optionally in a suitable solvent, with 1 to 6 equivalents of a carboxylic acid corresponding to the carboxylic acid anion R¹R²R³CCOO⁻.

29. A process for preparing an acyloxy compound according to claim 28, a solvent is present and the solvent comprises at least one member selected from the group consisting of alicyclic ethers, aliphatic and aliphatic difunctional ethers, esters, ketones, carbonates, nitriles, amines, acid amides, ionic liquids, water, alcohols, hydrocarbons, halogenated hydrocarbons, heterocyclic compounds and heteroaromatic compounds, or mixtures of at least two such solvents are used, preferentially diethyl ether, tetrahydrofuran, tetrahydro-2-methylfuran, tetrahydro-3-methylfuran, tetrahydro-2,5-dimethylfuran, tetrahydro-3,4-dimethylfuran, tetrahydropyran, cyclopentyl methyl ether, dimethoxyethane, diethoxymethane, diethoxyethane, polyethylene glycols, methanol, ethanol, propanol, isopropanol, butanol, isobutanol, tert-butanol, sec-butanol, 2-ethylhexanol, methyl formate, ethyl formate, propyl formate, methyl isobutyl ketone, methyl acetate, ethyl acetate, butyl acetate, methoxyethyl acetate, ethoxyethyl acetate, 2-(2-ethoxyethoxy)ethyl acetate, (2-methoxyethyl)methyl carbonate, ethylene carbonate, propylene carbonate, butylene carbonate, vinylene carbonate, dimethyl carbonate, diethyl carbonate, dipropyl carbonate, ethylmethyl carbonate, methylpropyl carbonate, butylmethyl carbonate, ethylpropyl-carbonate, butylethyl carbonate, γ-butyrolactone, γ-valerolactone, dimethylformamide, dimethylacetamide, dimethyl sulfoxide, triethylamine, piperidine, pyridine, acetonitrile, propionitrile, glutarodinitrile, adiponitrile, methoxypropionitrile, pentane, hexane, cyclohexane, heptane, methylcyclohexane, octane, benzene, toluene, a xylene, ethylbenzene, cumene, dichloromethane, chloroform and 1,2-dichloroethane.

30. A process for preparing an acyloxy compound, according to claim 28, wherein the proportion of the solvent in the course of the synthesis amounts to 0% to 70%.

31. A process for preparing an acyloxy compound according to claim 28, wherein the monitoring of the reaction is undertaken by means of IR spectroscopic tracking or turbidity measurement or particle-size measurement or conductivity measurement directly in the reaction vessel.

32. A method comprising performing an organic synthesis with an acyloxy compound according to claim 20.

33. A method comprising performing a chemical reaction by adding an acyloxy compound according to claim 20 as a reducing agent.

34. The method of claim 33, wherein the method is conducted in the presence of a carboxylic acid.

35. The method of claim 33, wherein the compound reduced is an oxo compound selected from the group consisting of a ketone, an aldehyde, an ester and an imines.

36. A method comprising performing a reductive animation of an oxo compound by with a compound according to claim 20,

37. A method comprising reducing a halide with the compound of claim 20.

38. A method comprising performing a chemical reaction, wherein an acyloxy compound according to claim 20 is present as a stereoselective reducing agent.
